(19) United States
(12) Patent Application Publication
(10) Pub. No.: US 2019/0298578 A1
(43) Pub. Date: Oct. 3, 2019

(54) VENTED EMERGENCY WOUND DRESSINGS WITH ANTI-THROMBOGENIC LAYERS

(71) Applicants: Stephen Shulman, Ann Arbor, MI (US); Stuart Shulman, Ann Arbor, MI (US)

(72) Inventors: Stephen Shulman, Ann Arbor, MI (US); Stuart Shulman, Ann Arbor, MI (US)

(21) Appl. No.: 16/424,117

(22) Filed: May 28, 2019

Related U.S. Application Data
(63) Continuation-in-part of application No. 16/144,170, filed on Sep. 27, 2018.
Application No. 16/144,170 is a reissue of application No. 14/247,884, filed on Apr. 8, 2014, now Pat. No. 9,452,088.
Application No. 14/247,884 is a continuation-in-part of application No. 12/748,008, filed on Mar. 26, 2010, now abandoned.
(60) Provisional application No. 61/915,198, filed on Dec. 12, 2013.
(60) Provisional application No. 61/163,613, filed on Mar. 26, 2009.

(51) Int. Cl. A61F 13/02

(52) U.S. Cl. CPC A61F 13/0213; A61F 2013/00314

(57) ABSTRACT

Wound dressings provide a tenacious occlusive seal against the skin of a wearer, even in the presence of excessive blood or heavy perspiration. The preferred embodiment uses a hydrogel island providing superior hydrophilic gel adhesion. The product performs under extreme temperatures (i.e., 32-140° F.), and may be used to hold other dressings in place. Different “vented” embodiments are disclosed. A preferred structure includes a cover layer peripherally bonded to backing and hydrogel layers having a central aperture therethrough. The cover layer may have one or more holes, passages, tunnels or pockets to release pressure from a wound through the central aperture to the ambient environment. Layers that come in contact with blood products are preferably anti-thrombogenic to reduce or eliminate thrombi, or clotting that might interfere or obstruct venting. In particular, blood-contacting cover and cover/backing layers may use a thermoplastic polyurethane (TPU) film, more particularly a polyether TPU film.

### REFERENCE TO RELATED APPLICATIONS

This application is a Continuation-In-Part of U.S. patent application Ser. No. 16/144,170, filed Sep. 27, 2018, which is a Re-Issue application Ser. No. 14/247,884, which is a Continuation-In-Part of U.S. patent application Ser. No. 12/748,008, filed Mar. 26, 2010, which claims priority from U.S. Provisional Patent Application Ser. No. 61/163,613, filed Mar. 26, 2009, the entire content of each application being incorporated herein by reference.

### FIELD OF THE INVENTION

This invention relates generally to wound dressings and, in particular, to vented wound dressings that provide a unique combination of a hydrogel and an anti-thrombogenetic layer to form effective patches for emergency situations, including battlefield applications.

### BACKGROUND OF THE INVENTION

There are instances when an emergency wound dressing may be required not only to stop bleeding, but also to vent the transfer of gasses and/or liquids from the wound. Bullet wounds in battlefield situations represent one such need. Until now, however, there are few if any bandages that will remain affixed to a recipient given heat and moisture from weather, sweating, and so forth.

### SUMMARY OF THE INVENTION

This invention is a wound dressing that uses a novel combination of materials to provide a tenacious occlusive seal, even in the presence of excessive blood or heavy perspiration. The preferred embodiment combines an adhesive backing layer with a hydrogel layer providing superior hydrophilic gel adhesion. The product performs under extreme temperatures (i.e., 32-140° F.), and may be used to hold other dressings in place. The preferred embodiments may be entirely translucent, and include a large pull-tab for easy removal from a release layer. One or two dressings may be packaged in a pliable re-sealable protective pouch.

Different “vented” embodiments are disclosed. Preferred structures include a cover layer peripherally bonded to backing and hydrogel layers having a central aperture therethrough. The cover layer has one or more vent passages, and pressure is released through the central aperture and out these vent paths.

In preferred embodiments, layers that come in contact with blood products are preferably anti-thrombogenic to reduce or eliminate thrombi, or clotting that might interfere or obstruct the venting process. In particular, a blood-contacting cover and/or backing layers may use a thermoplastic polyurethane (TPU) film, more particularly a polyether TPU film.

### BRIEF DESCRIPTION OF THE DRAWINGS

FIG. 1 is a plan view drawing that illustrates a non-vented embodiment of the invention;

FIG. 2 is a drawing that depicts a vented structure;

FIG. 3 is a drawing that depicts a vented structure with an active status indicator;

FIG. 4 is a partially exploded view of a wound dressing assembly according to the invention;

FIG. 5 is a plan view drawing of a non-vented embodiment that shows one set of dimensions.

FIG. 6 is an exploded view drawing that illustrates the stacking of layers in a vented embodiment of the invention;

FIG. 7 is a plan view drawing of a vented embodiment that shows one set of dimensions;

FIG. 8 is a not-to-scale cross section depicting the way in which a venting structure operates;

FIG. 9 is a perspective view drawing that illustrates how vent passages need not extend through a cover layer;

FIG. 10 is a drawing that illustrates an embodiment of the invention that uses fewer layers;

FIG. 11 illustrates the alternative use of a one-way bicuspid or tricuspid valve;

FIG. 12A shows a pouch or sleeve into which one or more dressings may be placed until use, thereby forming a dressing/carrier “kit”; and

FIG. 12B depicts a way in which two dressings may be folded against one another using two joined and hinged backing layers.

### DETAILED DESCRIPTION OF THE PREFERRED EMBODIMENTS

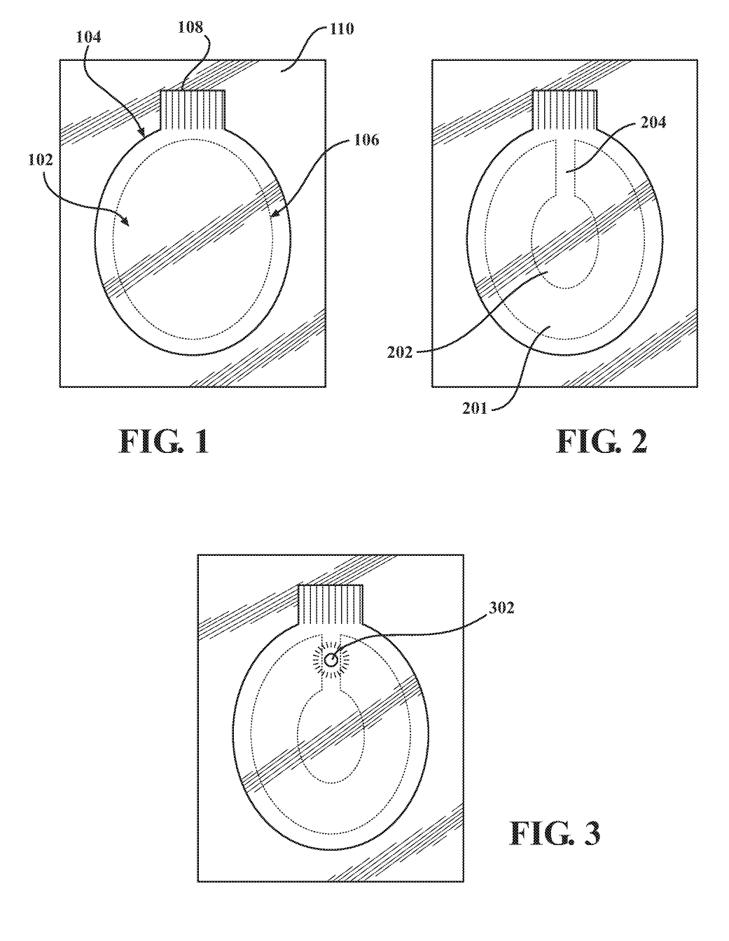

This invention is a wound dressing that uses a novel combination of materials to provide a tenacious occlusive seal, even in the presence of excessive blood or heavy perspiration. FIG. 1 is a plan view drawing that illustrates a non-vented embodiment of the invention, including a backing layer 104 and a pre-formed hydrogel pad 102. The backing layer 104 is preferably a flexible polyethylene translucent medical “tape” (i.e., 3.0 mil) having a hypoallergenic pressure-sensitive water-resistant adhesive such as an acrylic adhesive 500.

The hydrogel pad 102 may be obtained from Katecho, Inc. of Des Moines, Iowa (product code KM-10T). The backing material 110 is a polyester release liner (i.e., 3.0 mil), available from 3M Corp. of St. Paul, Minn., with a silicone coating on one side. Other vendors for the various materials are also possible. The structure further includes a clear, embossed tape medical tape piece 107 with a permanent adhesive, such as 3M 1527, bonded to a pull-tab 108, facilitating removal of the dressing from backing sheet 110.

The backing layer 104 may have a patient-facing adhesive layer, and the peripheral edge 106 of the backing layer may extend beyond the peripheral edge of the hydrogel pad 102 to expose this adhesive layer. Alternatively, backing layer 104 may not include an adhesive, in which case the peripheral edges of the backing layer 104 and the hydrogel pad 102 may be co-extensive. The release liner 110, however, preferaly extends beyond all sides of the backing layer 104, as shown in FIGS. 1-5.

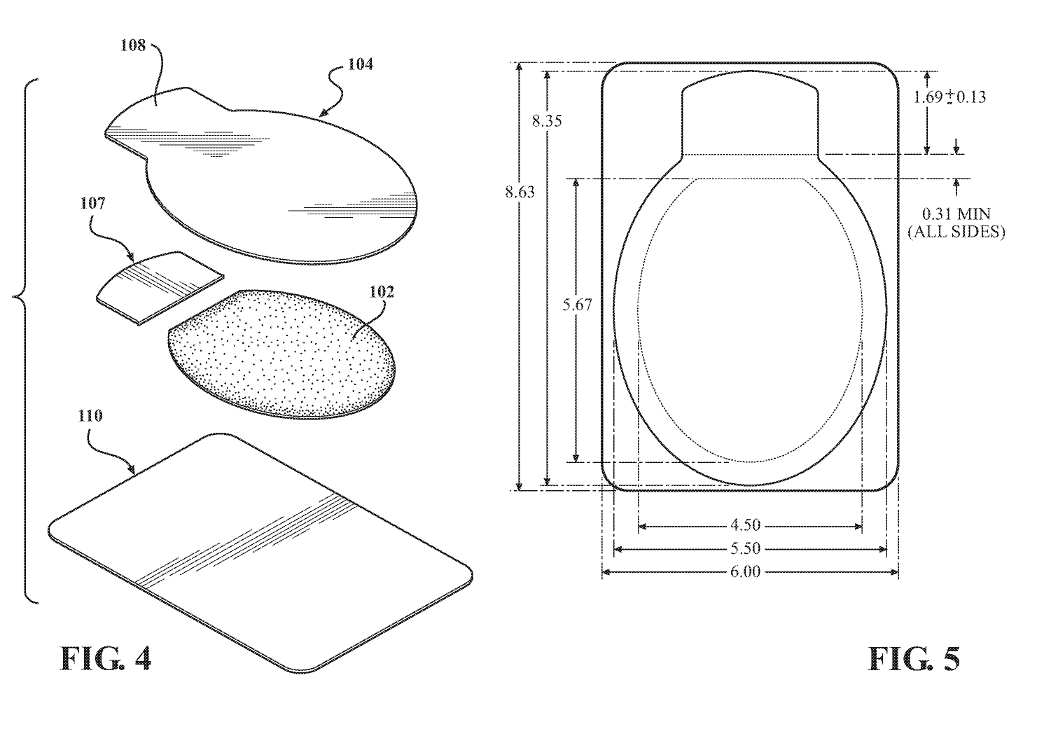

In terms of dimensions, the dressing has a preferred width on the order of 3 to 6 inches and a preferred length on the order of 4 to 8 inches. Other shapes besides ovals may be used, including circles, squares and rectangles. Materials other than polyethylene may be used for the backing layer 104 so long as the adhesive and combined system is sufficiently tenacious in terms of adherence to the skin. FIG. 4 is a partially exploded view of a wound dressing assembly according to the invention, and FIG. 5 is a plan view drawing that shows one set of dimensions.

On occasion, the wound dressing may need to vent pressure built up from a chest cavity, for example, and expell gasses or liquids, including blood products. To address this, one or more vents such as 204 may be provided as shown in FIG. 2. As pressure builds around a wound in region 202, it will be released through vent 204. In this case vent 204 comprises a gap in hydrogel layer 201, such that the vent 204 is a tunnel from region 202 to the ambient environment.

As a further alternative, a passive or active status sensor may be used to indicate a user condition or to show that pressure release has occurred. As shown in FIG. 3, such a monitor 302 may be passive, using dyes or other materials which change color in the presence of oxygen or other gasses, for example. The detector may be active, for example, using an electronic pressure sensor, an acoustic sensor (to detect lung sound) or resistive sensor (to detect perspiration or breathing) and a visual indicator such as an LED. The light may have different colors to convey status (i.e., green=OK; yellow=concern; red=emergency). In all embodiments, beneficial or therapeutic substances may be added to the hydrogel layer, including an antimicrobial such as chlorhexidine digluconate.

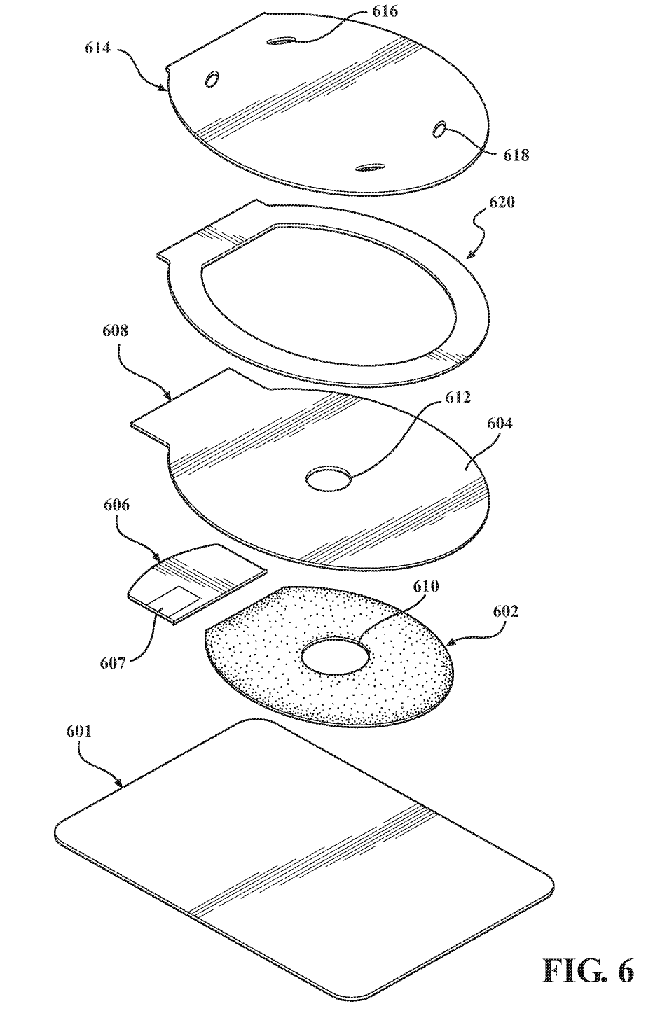

FIG. 6 is an exploded view drawing that illustrates a vented embodiment of the invention, showing the way in which the various layers are stacked. The article comprises a backing layer shown at 604, and the hydrogel pad is shown at 602. As with other embodiments disclosed herein, the backing layer is preferably a flexible polyethylene “tape” (i.e., 3.0 mil). The hydrogel material may be obtained from Katecho, Inc. of Des Moines, Iowa (product code KM-10T). The backing material may be obtained from 3M Corp. of St. Paul, Minn. as Part No. 1526. Again, other materials, and different vendors, are possible.

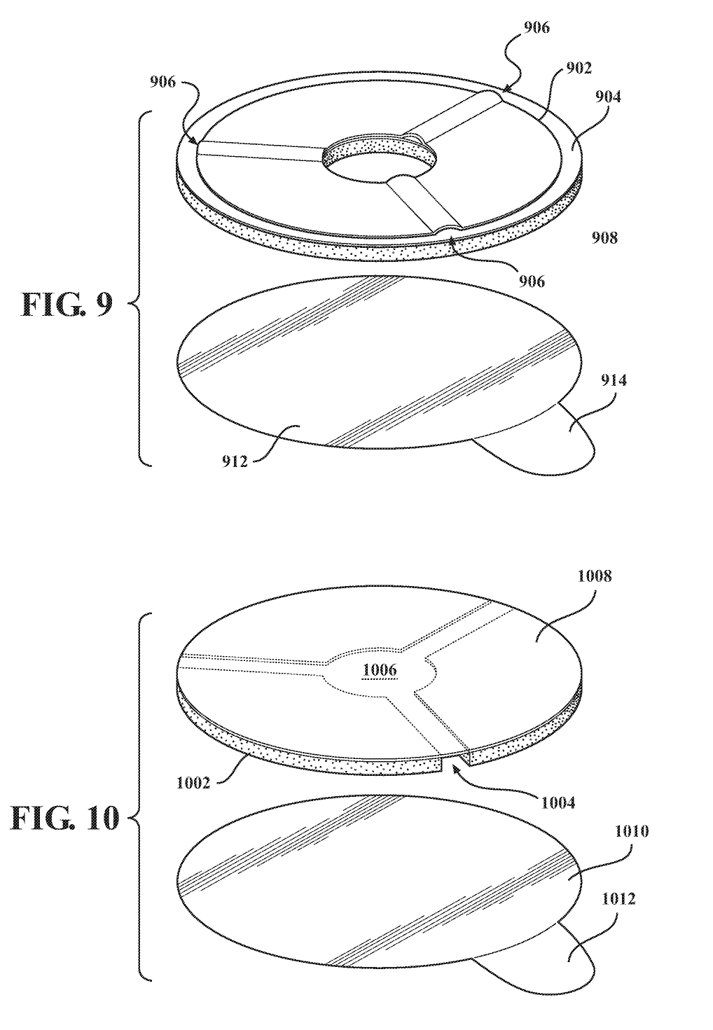

In the embodiment of FIG. 6, the hydrogel pad 602, which is preformed into the shape depicted, is adhered to the backing layer using an adhesive layer, the hydrogel itself, or both. The entire bottom surface of the backing layer 604 may be coated with an adhesive, such that once adhered, the hydrogel forms an island having a periphery spaced apart from the periphery of the backing layer 104 in all dimensions to expose the adhesive layer. Alternatively, as best seen in FIGS. 9, 10, the hydrogel layer need not extend beyond the periphery of the backing layer, in which case a separate adhesive layer between the hydrogel and the release layer may or may not be used.

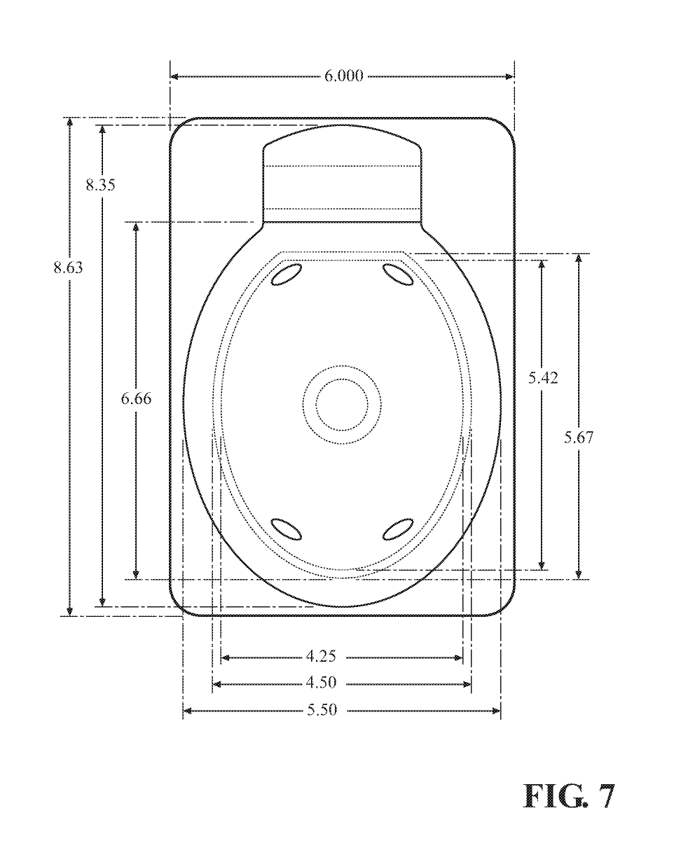

An adhesive-free release tab 608, uded to remove the dressing from release liner 601, may be provided by adhering a tape piece 606 to the tab 608. The adhesive-free release tab 608 may be printed (607) with product information. The release liner may be a polyester (i.e., 3.0 mil) film. A set of applicable dimensions for the vented product are provided in FIG. 7. Again, the dressing has a preferred width on the order of 3 to 6 inches and a preferred length on the order of 4 to 8 inches. Other shapes besides ovals may be used, including circles, squares and rectangles.

Continuing the reference to FIG. 6, the hydrogel layer 602 includes a central aperture 610, which is aligned with a central aperture 612 in backing layer 604. Both apertures are preferably round, with diameters in the range of 0.5 to 2.0 inches. As shown, the aperture 610 in the hydrogel layer 602 may be larger than aperture 612 in backing layer 604, or they may be the same size.

On the outer side of the backing layer, facing away from the recipient, there is attached a cover layer 614 having one or more vent holes 616, 618. The cover 614 is peripherally bonded to the outer surface of the backing layer using a ring-shaped polyester tape 620 having an adhesive on both sides. The cover 614 may also be attached directly to the backing layer with or without an adhesive, using thermal welding, for example.

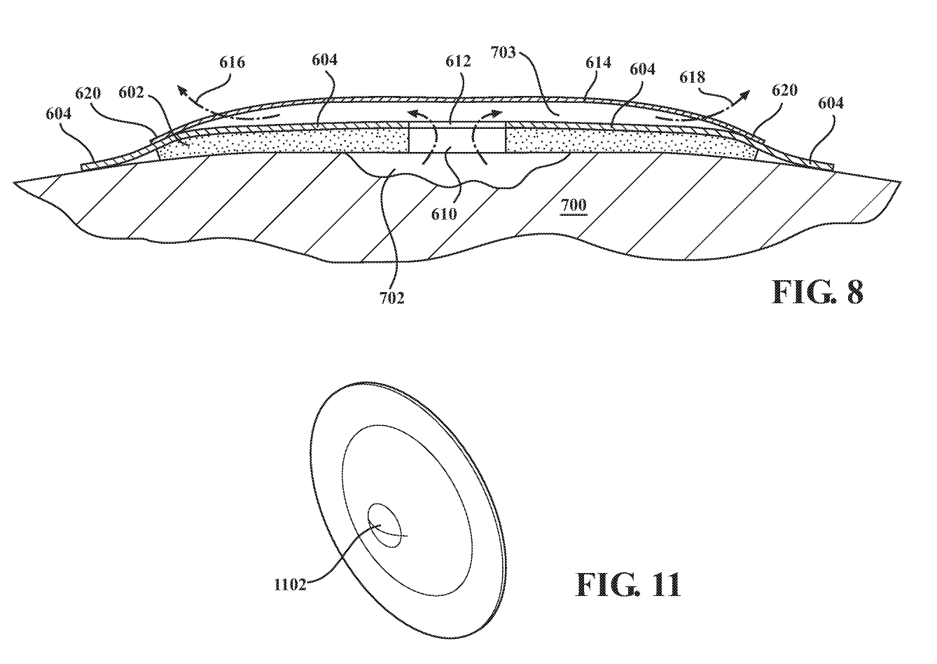

FIG. 8 is a not-to-scale cross section depicting the way in which the venting structure operates. As can be seen, if pressure builds up from wound 702 on patient 700, the gasses (or liquids) can flow through apertures 610, 612 and out to atmosphere through vent holes 616, 618. When this happens, the entire cover 614 may pull slightly away from the backing material 604, temporarily creating a pocket 703 that is depressurized through the vent holes. After the pressure is relieved or with negative pressure, the cover 614 will typically fall back onto backing layer 604, thereby maintaining a seal around the wound.

FIG. 9 further clarifies that the vent holes need not extend through the cover layer 902, but instead may be selectively adhered to backing layer 904 in the hatched areas to form one or more tunnels or channels 906 akin to the single channel 204 shown in FIG. 2. The backing layer 904 and hydrogel layer 908 have aligned apertures 910, whereby the tunnels 906 lead to this central region. Note that the cover layer 902 is depicted as a transparent layer to visualize the central aperture(s) 910. The release liner 912 may include pull tab 914. The backing layer 904 may be may or may not have an exposed adhesive, but may instead rely only on hydrogel layer 908 for adhesion, in which case the hydrogel and backing layer may be the same size. The cover layer 902 may be the same size as the backing layer 904, or may be smaller, as shown.

FIG. 10 is a drawing that illustrates an embodiment of the invention that uses fewer layers. To accomplish this, the hydrogel layer 1002 has one of more gaps or channels 1004 leading to a central region 1006. In this case the backing layer 1008 essentially forms the “cover.” Again, the hydrogel layer 1002 may extend all the way to the edge of the backing layer 1008, such that layer 1008 may not include a separate adhesive.

As yet a further embodiment, as opposed to an open central aperture through the hydrogel and backing layers leading to a pocket formed with the cover layer, a one-way bicuspid 1102 or tricuspid valve of the type shown in FIG. 11 may be used. The apex of the valve would be oriented away from the recipient, which would open when pressurized, and close following pressurization to maintain cleanliness.

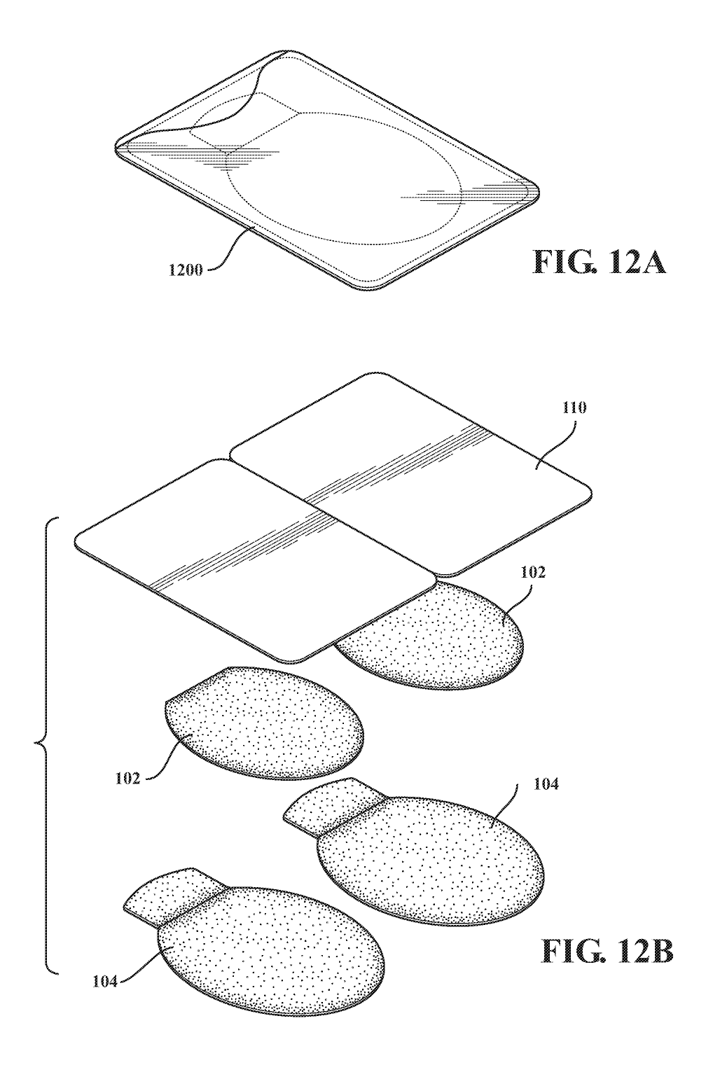

FIGS. 12A, B illustrate packaging techniques according to the invention, for either the non-vented or vented embodiments. FIG. 12A shows a pouch or sleeve 1200 into which a dressing may be placed until use, thereby forming a dressing/carrier “kit.” As shown in FIG. 12B, two dressings may be folded against one another using two joined and hinged backing layers 110. Pouch or sleeve 1200 may then receive the two connected dressings. As yet a further option, pouch or sleeve 1200 may also be re-sealable.

### Anti-Thrombogenicity

In all embodiments of this invention, layers that come in contact with blood products are preferably anti-thrombogenic to reduce or eliminate thrombi, or clotting. Thus, in embodiments that include a cover layer, such as layer 614 in FIGS. 6-8 and 902 in FIG. 9, these cover layers are preferably anti-thrombogenic. In embodiments wherein the backing layer serves as the cover layer, as with layer 1008 in FIG. 10, this layer is preferably anti-thrombogenic.

Thrombogenicity refers to the tendency of a material in contact with the blood to produce a thrombus, or clot. The term not only refers to fixed thrombi but also to emboli, thrombi that have become detached and travel through the bloodstream. Embodiments of this invention incorporate anti-thrombogenic blood-contacting layers because coagulation and thrombosis caused by platelet adhesion and activation on material surfaces may lead to functional failure and even fatal outcomes.

Blood-contacting cover and cover/backing layers may use a thermoplastic polyurethane (TPU) film, more particularly a polyether TPU film. More specifically, a product such as WPF 24-025 ABC may be used, a 2.5-mil polyether polyurethane film available from BASF, Inc. that exhibits anti-thrombogenic properties. It is believed that the anti-thrombogenic properties of such polyether TPU films are directly correlated to the MVTR (moisture vapor transmission rate) of the TPU chemistry—a higher MVTR results in higher anti-thrombogenicity.

1. A vented wound dressing, comprising:
a hydrogel layer having inner and outer surfaces, and wherein the inner surface is adapted skin contact and adherence;
a cover or backing layer adhered to the outer surface of the hydrogel layer;
a release layer temporarily affixed to the inner surface of the backing layer, and wherein the release layer is removed for placement onto the skin such that the central aperture is aligned with a wound;
one or more paths between the cover or backing layer and the hydrogel layer enabling gasses or liquids to vent from the wound and central aperture to the ambient environment; and
wherein the cover or backing layer is composed of an anti-thrombogenic material.

2. The vented wound dressing of claim 1, wherein the cover or backing layer is a polyether polyurethane film.

3. The vented wound dressing of claim 1, including a single cover or backing layer; and
wherein the one or more paths are formed with gaps in the hydrogel layer leading from the central aperture to the ambient environment.

4. The vented wound dressing of claim 1, including separate cover and backing layers; and
wherein at least a portion of the one or more paths is formed by a space between the separate cover and backing layers.

5. The vented wound dressing of claim 4, wherein the backing layer includes a central aperture aligned with central aperture in the hydrogel layer; and
wherein the one or more paths between the cover and backing layers are channels or tunnels formed through selective bonding of the cover layer to the backing layer.

6. The vented wound dressing of claim 4, wherein the backing layer includes a central aperture aligned with central aperture in the hydrogel layer;
wherein the cover layer is peripherally bonded to the backing layer; and
the one or more paths between the cover and backing layers lead to one more holes in the cover layer.

7. The vented wound dressing of claim 1, further including a pouch to protect the dressing, thereby providing a portable kit.

8. The vented wound dressing of claim 1, having an oval or circular shape.

9. The vented wound dressing of claim 1, having a shape that is rectangular.

10. The vented wound dressing of claim 1, having a width in the range of 3 to 6 inches.

11. The vented wound dressing of claim 1, having a length in the range of 4 to 8 inches.
